(19) United States
(12) Patent Application Publication
(10) Pub. No.: US 2024/0110135 A1
(43) Pub. Date: Apr. 4, 2024

(54) MICRO-ORGAN SPECIFICITY MAINTENANCE SYSTEM

(71) Applicants: Yulin CAO, Jiyuan (CN); Tangyi Holding (Shenzhen) Co., Ltd., Shenzhen (CN)

(72) Inventor: Yulin CAO, Beijing (CN)

(73) Assignees: Yulin CAO, Jiyuan (CN); Tangyi Holding (Shenzhen) Co., Ltd., Shenzhen (CN)

(21) Appl. No.: 18/084,588

(22) Filed: Dec. 20, 2022

(30) Foreign Application Priority Data
Sep. 30, 2022 (CN) 202211207956.X

(51) Int. Cl. C12M 3/00; A01N 1/02; C12M 1/00; C12M 1/34; C12M 1/36; G01N 33/50

(52) U.S. Cl. CPC C12M 21/08; A01N 1/0247; C12M 23/44; C12M 29/10; C12M 41/32; C12M 41/48; G01N 33/5088

(57) ABSTRACT

A micro-organ specificity maintenance system includes a micro-organ specificity parallel reactor module for storing and culturing vascularized micro-organs; a feeding module for feeding culture solutions to the micro-organ specificity parallel reactor module; an injection module for injecting medical solutions containing traditional Chinese medicine micromolecules into the micro-organ specificity parallel reactor module; a micro-organ circulation coupling module for transmitting venous flow and arterial flow to the micro-organ specificity parallel reactor module; and a micro-organ neural coupling module for transmitting neural network signals to the micro-organ specificity parallel reactor module. The micro-organ specificity maintenance system provided in the disclosure has the advantage that an extracorporeal circulation system and internal vascular systems of complex organs are organically integrated and connected.

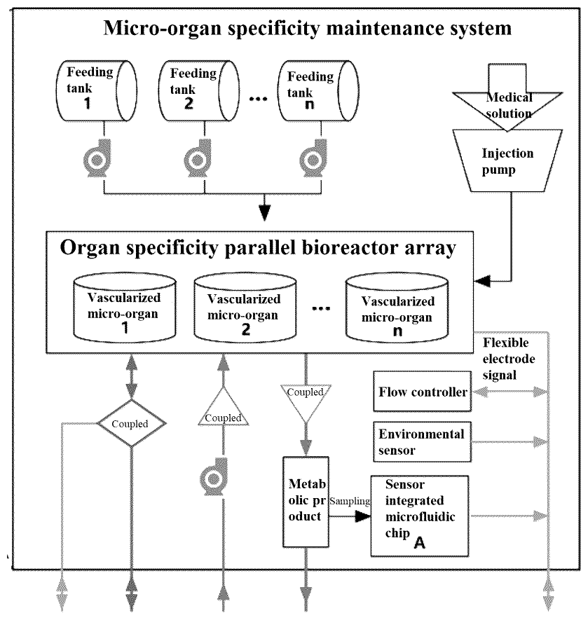

### CROSS REFERENCE TO THE RELATED APPLICATIONS

This application is based upon and claims priority to Chinese Patent Application No. 202211207956.X, filed on Sep. 30, 2022, the entire contents of which are incorporated herein by reference.

### TECHNICAL FIELD

The disclosure relates to the technical field of extracorporeal organ maintenance, and particularly to a micro-organ specificity maintenance system.

### BACKGROUND

In vitro culture, a biological term, means a method through which living structural components (such as living tissue, living cells, living organs, etc.) and even living individuals are taken out of a body or a host and then placed in an in vitro environment similar to the living environment in vivo for growth and development.

When the existing extracorporeal organ maintenance system is used, the single organ is cultured and maintained, and the information communication between a plurality of organs cannot be realized, thereby influencing the organic integration and connection of the extracorporeal circulation system and the internal vascular systems of complex organs.

Therefore, it is necessary to provide a novel micro-organ specificity maintenance system to solve the above technical problems.

### SUMMARY

In order to solve the above technical problems, the disclosure provides a micro-organ specificity maintenance system for organic integration and connection of an extracorporeal circulation system and internal vascular systems of complex organs.

A micro-organ specificity maintenance system provided in the disclosure includes: a micro-organ specificity parallel reactor module for maintaining and culturing micro-organs configured by a plurality of consistent vascularized micro-organ reactors according to specific culture environments of the micro-organs; a feeding module for feeding culture solutions to the micro-organ specificity parallel reactor module; an injection module for injecting medical solutions containing traditional Chinese medicine micromolecules into the micro-organ specificity parallel reactor module; a micro-organ circulation coupling module for starting and closing venous flow and arterial flow for the micro-organ specificity parallel reactor module; a micro-organ neural coupling module for transmitting and closing electroneurographic signals for the micro-organ specificity parallel reactor module; and a signal detection and control module for monitoring flow towards the micro-organ specificity parallel reactor module and an environment of the micro-organ specificity parallel reactor module, and further for sampling and testing venous blood after metabolic products of the micro-organs enter the venous flow. The micro-organ specificity parallel reactor module is in communication with the feeding module and the injection module. The micro-organ specificity parallel reactor module and the micro-organs are connected to a nervous system in a two-way manner by means of the micro-organ neural coupling module, such that electroneurographic signals are only allowed to be transmitted in from the nervous system, neural signals of the micro-organs are only allowed to be transmitted out, or neural signals generated by the nervous system and the micro-organs are allowed to be transmitted in a two-way manner. The micro-organ specificity parallel reactor module is connected to an arteriovenous vascular system in a one-way manner by means of the micro-organ circulation coupling module, and the micro-organ circulation coupling module is a one-way switch for controlling inflow and outflow of the arterial flow and the venous flow. The micro-organ specificity parallel reactor module is connected to the signal detection and control module by means of flexible electrode signals, so as to inspect and control internal environment states of micro-organ reactors and automatically adjust control parameters.

Preferably, the micro-organ specificity parallel reactor module includes the micro-organ reactors in which the vascularized micro-organs are cultured. According to micro-organ specificity survival periods and usage periods required by experiments, a plurality of consistent micro-organ reactors are placed according to survival period intervals for time-progressive culture, and according to data measured by the signal detection and control module, connection and disconnection between a certain micro-organ reactor and the injection module, the micro-organ circulation coupling module, the micro-organ neural coupling module and the signal detection and control module are automatically switched.

Preferably, the micro-organ circulation coupling module includes an arterial flow assembly and a venous flow assembly. The arterial flow assembly includes a micro-flow pump and a micro-organ circulation arterial flow coupling device, an input end of the micro-flow pump is connected to an external one-way arterial line, an output end of the micro-flow pump is in communication with an input end of the micro-organ circulation arterial flow coupling device, and an output end of the micro-organ circulation arterial flow coupling device is connected to arterial input ends of the vascularized micro-organs in the micro-organ reactors in a one-way manner. The venous flow assembly includes a micro-organ circulation venous flow coupling device, an input end of the micro-organ circulation venous flow coupling device is connected to venous output ends of the vascularized micro-organs in the micro-organ reactors, and an output end of the micro-organ circulation venous flow coupling device is in communication with an external one-way venous line.

Preferably, the micro-organ neural coupling module includes a micro-organ circulation neural coupling device, one end of the micro-organ circulation neural coupling device is connected to an external human brain micro-organ specificity maintenance system, one end of the micro-organ circulation neural coupling device is connected to neurons of the vascularized micro-organs in the micro-organ reactors, and the micro-organ circulation neural coupling device is further connected to an external signal communication node.

Preferably, the feeding module includes a feeding tank, the feeding tank is in communication with the micro-organ reactors by means of micro-flow pumps, and substances such as culture solutions are injected into the micro-organ reactors by means of the feeding tank.

Preferably, the injection module includes an injection pump, a liquid outlet of the injection pump is connected to medicine inlets of the micro-organ reactors, and the medical solutions containing traditional Chinese medicine micromolecules are injected into the micro-organ reactors by means of the injection pump.

Preferably, the signal detection and control module includes a flow controller, an environmental sensor and a sensor integrated microfluidic chip, the flow controller, the environmental sensor and the sensor integrated microfluidic chip are connected to the micro-organ reactors by means of the flexible electrode signals, the sensor integrated microfluidic chip is further in communication with an output end of the micro-organ circulation venous flow coupling device, and the metabolic products in the venous flow flowing out from the output end of the micro-organ circulation venous flow coupling device are sampled and tested by means of the sensor integrated microfluidic chip.

Compared with the prior art, the micro-organ specificity maintenance system provided in the disclosure has the beneficial effects as follows:

According to the disclosure, by injecting the medical solutions containing traditional Chinese medicine micromolecule into the micro-organ specificity parallel reactor module, structures and functions of complex organs are effectively promoted to be mature and maintenance culture periods of the complex organs are widened. Moreover, in-situ, three-dimensional and multi-point collection of bioelectric signals and biochemical signals of the complex organs is realized by means of the micro-organ circulation coupling module, the micro-organ neural coupling module and the signal detection and control module.

According to the disclosure, a microphysiological system is constructed to simulate in-vitro interaction of the complex organs of human blood circulation and nerve regulation and control by means of the micro-organ circulation coupling module, the micro-organ neural coupling module and the signal detection and control module: through integration of biology and engineering, a traditional organ-like culture mode is broken through, and an extracorporeal circulation system and internal vascular systems of the complex organs are organically integrated and connected to realize perfusion and metabolic circulation; biosensing and signal positive feedback transmission are used for information communication between multi-micro-organs; and by means of a controllable circulation device, a controllable “switch” is mounted to realize interaction and function regulation and control between any organs.

### BRIEF DESCRIPTION OF THE DRAWINGS

FIGURE is a schematic structural diagram of a process of a micro-organ specificity maintenance system provided in the disclosure.

### DETAILED DESCRIPTION OF THE EMBODIMENTS

The disclosure is further described below in combination with accompanying drawings and embodiments.

With reference to the FIGURE, a micro-organ specificity maintenance system includes: a micro-organ specificity parallel reactor module for maintaining and culturing micro-organs configured by a plurality of consistent vascularized micro-organ reactors according to specific culture environments of the micro-organs; a feeding module for feeding culture solutions to the micro-organ specificity parallel reactor module; an injection module for injecting medical solutions containing traditional Chinese medicine micromolecules into the micro-organ specificity parallel reactor module; a micro-organ circulation coupling module for starting and closing venous flow and arterial flow for the micro-organ specificity parallel reactor module; a micro-organ neural coupling module for transmitting and closing electroneurographic signals to the micro-organ specificity parallel reactor module; and a signal detection and control module for monitoring flow towards the micro-organ specificity parallel reactor module and an environment of the micro-organ specificity parallel reactor module, and further for sampling and testing venous blood after metabolic products of the micro-organs enter the venous flow. The micro-organ specificity parallel reactor module is in communication with the feeding module and the injection module. The micro-organ specificity parallel reactor module and the micro-organs are connected to a nervous system in a two-way manner by means of the micro-organ neural coupling module, such that electroneurographic signals are only allowed to be transmitted in from the nervous system, neural signals of the micro-organs are only allowed to be transmitted out, or neural signals generated by the nervous system and the micro-organs are allowed to be transmitted in a two-way manner. The micro-organ specificity parallel reactor module is connected to an arteriovenous vascular system in a one-way manner by means of the micro-organ circulation coupling module, and the micro-organ circulation coupling module is a one-way switch for controlling inflow and outflow of the arterial flow and the venous flow. The micro-organ specificity parallel reactor module is connected to the signal detection and control module by means of flexible electrode signals, so as to inspect and control internal environment states of the micro-organ reactors and automatically adjust control parameters.

It should be noted that by injecting the medical solutions containing traditional Chinese medicine micromolecule into the micro-organ specificity parallel reactor module, structures and functions of complex organs are effectively promoted to be mature and maintenance culture periods of the complex organs are widened. Moreover, in-situ, three-dimensional and multi-point collection of bioelectric signals and biochemical signals of the complex organs is realized by means of the micro-organ circulation coupling module, the micro-organ neural coupling module and the signal detection and control module.

It should be noted that a microphysiological system is constructed to simulate in-vitro interaction of the complex organs of human blood circulation and nerve regulation and control by means of the micro-organ circulation coupling module, the micro-organ neural coupling module and the signal detection and control module: through integration of biology and engineering, a traditional organ-like culture mode is broken through, and an extracorporeal circulation system and internal vascular systems of the complex organs are organically integrated and connected to realize perfusion and metabolic circulation; biosensing and signal positive feedback transmission are used for information communication between multi-micro-organs; and by means of a controllable circulation device, a controllable “switch” is mounted to realize interaction and function regulation and control between any organs.

The micro-organ specificity parallel reactor module includes the micro-organ reactors in which the vascularized micro-organs are cultured. According to micro-organ specificity survival periods and usage periods required by experiments, a plurality of consistent micro-organ reactors are placed according to survival period intervals for time-progressive culture, and according to data measured by the signal detection and control module, connection and disconnection between a certain micro-organ reactor and the injection module, the micro-organ circulation coupling module, the micro-organ neural coupling module and the signal detection and control module are automatically switched.

The micro-organ circulation coupling module includes an arterial flow assembly and a venous flow assembly. The arterial flow assembly includes a micro-flow pump and a micro-organ circulation arterial flow coupling device, an input end of the micro-flow pump is connected to an external one-way arterial line, an output end of the micro-flow pump is in communication with an input end of the micro-organ circulation arterial flow coupling device, and an output end of the micro-organ circulation arterial flow coupling device is connected to arterial input ends of the vascularized micro-organs in the micro-organ reactors in a one-way manner. The venous flow assembly includes a micro-organ circulation venous flow coupling device, an input end of the micro-organ circulation venous flow coupling device is connected to venous output ends of the vascularized micro-organs in the micro-organ reactors, and an output end of the micro-organ circulation venous flow coupling device is in communication with an external one-way venous line.

The micro-organ neural coupling module includes a micro-organ circulation neural coupling device, one end of the micro-organ circulation neural coupling device is connected to an external human brain micro-organ specificity maintenance system, one end of the micro-organ circulation neural coupling device is connected to neurons of the vascularized micro-organs in the micro-organ reactors, and the micro-organ circulation neural coupling device is further connected to an external signal communication node.

The feeding module includes a feeding tank, the feeding tank is in communication with the micro-organ reactors by means of micro-flow pumps, and substances such as culture solutions are injected into the micro-organ reactors by means of the feeding tank.

The injection module includes an injection pump, a liquid outlet of the injection pump is connected to medicine inlets of the micro-organ reactors, and the medical solutions containing traditional Chinese medicine micromolecules are injected into the micro-organ reactors by means of the injection pump.

The signal detection and control module includes a flow controller, an environmental sensor and a sensor integrated microfluidic chip, the flow controller, the environmental sensor and the sensor integrated microfluidic chip are connected to the micro-organ reactors by means of the flexible electrode signals, the sensor integrated microfluidic chip is further in communication with an output end of the micro-organ circulation venous flow coupling device, and the metabolic products in the venous flow flowing out from the output end of the micro-organ circulation venous flow coupling device are sampled and tested by means of the sensor integrated microfluidic chip.

What is described above is only the examples of the disclosure and does not limit the patent scope of the disclosure, and equivalent structures or equivalent process changes made by means of the contents of the description and the accompanying drawings of the disclosure, or directly or indirectly used in other related technical fields shall all fall within the scope of patent protection of the disclosure in a similar way.

What is claimed is:

1. A micro-organ specificity maintenance system, comprising:
a micro-organ specificity parallel reactor module for maintaining and culturing micro-organs configured by a plurality of consistent vascularized micro-organ reactors according to specific culture environments of the micro-organs;
a feeding module for feeding culture solutions to the micro-organ specificity parallel reactor module;
an injection module for injecting medical solutions containing traditional Chinese medicine micromolecules into the micro-organ specificity parallel reactor module;
a micro-organ circulation coupling module for starting and closing a venous flow and an arterial flow for the micro-organ specificity parallel reactor module;
a micro-organ neural coupling module for transmitting and closing electroneurographic signals for the micro-organ specificity parallel reactor module; and
a signal detection and control module for monitoring a flow towards the micro-organ specificity parallel reactor module and an environment of the micro-organ specificity parallel reactor module, and further for sampling and testing a venous blood after metabolic products of the micro-organs enter the venous flow; wherein
the micro-organ specificity parallel reactor module is in a communication with the feeding module and the injection module; the micro-organ specificity parallel reactor module and the micro-organs are connected to a nervous system in a two-way manner by the micro-organ neural coupling module, such that electroneurographic signals are only allowed to be transmitted in from the nervous system, neural signals of the micro-organs are only allowed to be transmitted out, or neural signals generated by the nervous system and the micro-organs are allowed to be transmitted in the two-way manner; the micro-organ specificity parallel reactor module is connected to an arteriovenous vascular system in a one-way manner by the micro-organ circulation coupling module, and the micro-organ circulation coupling module is a one-way switch for controlling inflow and outflow of the arterial flow and the venous flow; and the micro-organ specificity parallel reactor module is connected to the signal detection and control module by flexible electrode signals, so as to inspect and control internal environment states of the plurality of consistent vascularized micro-organ reactors and automatically adjust control parameters.

2. The micro-organ specificity maintenance system according to claim 1, wherein the micro-organ specificity parallel reactor module comprises the plurality of consistent vascularized micro-organ reactors, vascularized micro-organs are cultured in the plurality of consistent vascularized micro-organ reactors; and according to micro-organ specificity survival periods and usage periods required by experiments, a plurality of consistent micro-organ reactors are placed according to survival period intervals for a time-progressive culture, and according to a data measured by the signal detection and control module, a connection and a disconnection between a predetermined micro-organ reactor of the plurality of consistent vascularized micro-organ reactors and the injection module, the micro-organ circulation coupling module, the micro-organ neural coupling module, and the signal detection and control module are automatically switched.

3. The micro-organ specificity maintenance system according to claim 1, wherein the micro-organ circulation coupling module comprises an arterial flow assembly and a venous flow assembly; the arterial flow assembly comprises a micro-flow pump and a micro-organ circulation arterial flow coupling device, an input end of the micro-flow pump is connected to an external one-way arterial line, an output end of the micro-flow pump is in a communication with an input end of the micro-organ circulation arterial flow coupling device, and an output end of the micro-organ circulation arterial flow coupling device is connected to arterial input ends of vascularized micro-organs in the plurality of consistent vascularized micro-organ reactors in the one-way manner; and the venous flow assembly comprises a micro-organ circulation venous flow coupling device, an input end of the micro-organ circulation venous flow coupling device is connected to venous output ends of the vascularized micro-organs in the plurality of consistent vascularized micro-organ reactors, and an output end of the micro-organ circulation venous flow coupling device is in a communication with an external one-way venous line.

4. The micro-organ specificity maintenance system according to claim 1, wherein the micro-organ neural coupling module comprises a micro-organ circulation neural coupling device, a first end of the micro-organ circulation neural coupling device is connected to an external human brain micro-organ specificity maintenance system, a second of the micro-organ circulation neural coupling device is connected to neurons of vascularized micro-organs in the plurality of consistent vascularized micro-organ reactors, and the micro-organ circulation neural coupling device is further connected to an external signal communication node.

5. The micro-organ specificity maintenance system according to claim 1, wherein the feeding module comprises a feeding tank, the feeding tank is in a communication with the plurality of consistent vascularized micro-organ reactors by micro-flow pumps, and substances such as culture solutions are injected into the plurality of consistent vascularized micro-organ reactors by the feeding tank.

6. The micro-organ specificity maintenance system according to claim 1, wherein the injection module comprises an injection pump, a liquid outlet of the injection pump is connected to medicine inlets of the plurality of consistent vascularized micro-organ reactors, and the medical solutions containing traditional Chinese medicine micromolecules are injected into the plurality of consistent vascularized micro-organ reactors by the injection pump.

7. The micro-organ specificity maintenance system according to claim 1, wherein the signal detection and control module comprises a flow controller, an environmental sensor, and a sensor integrated microfluidic chip, the flow controller, the environmental sensor, and the sensor integrated microfluidic chip are connected to the plurality of consistent vascularized micro-organ reactors by the flexible electrode signals, the sensor integrated microfluidic chip is further in a communication with an output end of the micro-organ circulation venous flow coupling device, and metabolic products in the venous flow flowing out from the output end of the micro-organ circulation venous flow coupling device are sampled and tested by the sensor integrated microfluidic chip.
